(19) United States
(12) Patent Application Publication
(10) Pub. No.: US 2022/0186312 A1
(43) Pub. Date: Jun. 16, 2022

(54) PREDICTIVE METHOD FOR ASSESSING THE SUCCESS OF EMBRYO IMPLANTATION

(71) Applicant: Guzip Biomarkers Corporation, Hsinchu County (TW)

(72) Inventors: Hung-Cheng Lai, TAIPEI CITY (TW); Rui-Lan Huang, New Taipei City (TW); Shu-Chuan Lin, New Taipei City (TW)

(73) Assignee: Guzip Biomarkers Corporation, Hsinchu County (TW)

(21) Appl. No.: 17/547,232

(22) Filed: Dec. 10, 2021

Related U.S. Application Data
(60) Provisional application No. 63/124,097, filed on Dec. 11, 2020.

(51) Int. Cl. C12Q 1/6876; G16B 40/00

(52) U.S. Cl. CPC C12Q 1/6876; C12Q 2600/158; C12Q 2600/154; G16B 40/00

(57) ABSTRACT

A method for identifying a potential biomarker for determining the probability of the success of embryo implantation by assaying a methylation profile of cervical secretions.

### FIELD OF THE INVENTION

The present invention relates a method for assessing endometrial receptivity of a female subject before embryo implantation, comprising performing an assay on fertility-associated biomarkers in methylation profiles of cervical secretions of the female subject.

### BACKGROUND OF THE INVENTION

In vitro fertilization (IVF) has become the most effective treatment for women who have 59 difficulties conceiving since the first baby was born via this medically assisted reproduction method in 1978. The number of IVF treatments performed is continuing to increase globally. A successful pregnancy relies on embryo, endometrium and embryo-endometrium synchronization. Although the selection of euploid embryos has been achieved via the application of preimplantation genetic testing for aneuploidies (PGT-A), resulting in increased clinical pregnancy rates and live birth rates, favorable outcomes after the transfer of embryos are not always guaranteed. Ovulation induction protocols and embryo culture systems in the laboratory have been continuously optimized following decades of development, resulting in improved quantity and quality of embryos. However, the implantation rate remains 25-40%, preventing IVF from having an ideal outcome. To overcome the last barrier to IVF success, namely, the implantation process, endometrial status must become readily assessable.

Implantation requires highly orchestrated interactions between the developing embryo and endometrium. The association between abnormal implantation and reproductive failure is evident. The ability of the endometrium to allow implantation of the embryo is termed receptivity. A successful pregnancy must be established on a receptive endometrium. Although efforts have been made to characterize a receptive endometrium, neither morphological parameters nor molecular biomarkers correlate well with pregnancy outcomes. Normal implantation occurs during a short time period in the mid-secretory phase termed the window of implantation (WOI). In this period, the endometrium becomes optimally receptive to support embryo implantation. Recently, a transcriptomic profile based on endometrial biopsies suggested that implantation failure results from displacement of the WOI. In addition, according to a transcriptomic analysis, pregnancy can be achieved if the timing of embryo transfer is advanced or delayed. Identifying the timeframe of the WOI can improve pregnancy outcomes in IVF by optimizing the synchrony between embryo and endometrium. However, implantation failure is more common for an endometrium with abnormal or absent WOI.

The human endometrium is a unique tissue that undergoes monthly changes involving regeneration, remodeling, and degradation. In each cycle, endometrial stem/progenitor cells are responsible for construction of the new endometrium following shedding of the old one. The substantial rearrangement of endometrial tissue during the menstrual phase is accompanied by vigorous epigenetic alterations. The DNA methylation of the endometrium then remains almost unchanged through the menstrual cycle until the late-secretory phase when the endometrium starts to break down. DNA methylation is a major epigenetic event involving the addition of a methyl group (—CH₃) to the carbon at position 5 of cytosine residues in the DNA template. Aberrant methylation of promoter regions of several genes has been found to be strongly associated with diseases. Since DNA methylation of the endometrium drastically changes only when stem/progenitor cells participate in the regeneration, it is likely that each newly grown endometrium has a distinct DNA methylation landscape regulating its behaviors, including the ability to allow embryo implantation. As evidenced by several studies, alterations in DNA methylation impair the expression of genes involved in embryo-endometrium crosstalk, implantation, and decidualization, leading to low fecundity. Evidence also indicates that the DNA methylome of endometrial tissue differs between healthy fertile donors and women suffering recurrent implantation failure. So far, most studies investigating the receptivity of the endometrium have been based on analysis of endometrial tissue obtained through biopsies. Endometrial biopsy is a blind & invasive procedure done by inserting a thin catheter through the natural opening of the cervix and into the uterine cavity to sample the endometrial cavity. In an endometrial biopsy, a small piece of tissue from the lining of the uterus is removed. Since the invasiveness of endometrial biopsies is detrimental to embryo implantation, embryos must be transferred in cycles separate from the analyzed one. Therefore, differences in the endometrium between different menstrual cycles cannot be evaluated by invasive approaches and are thus always ignored. Criticisms of invasive analysis such as inconsistent results being obtained between menstrual cycles in the same individual and inconclusive benefits of personalized embryo transfer based on a transcriptome-defined WOI might be explained by monthly variation of the endometrium.

From experience in cancer screening, cancer-associated DNA methylation can be detected in cell-free DNA or fragmented DNA present in body fluids and secretions. Indeed, the DNA methylome in cervical scrapings has been used as a noninvasive biomarker for the detection of endometrial cancer with high accuracy. Because cervical secretions can reflect the intrauterine environment, methylation profiles may be used as proxies for investigating the differences of DNA methylome in the endometrium between pregnancy and non-pregnancy cycles.

### SUMMARY OF THE INVENTION

The present invention provides a predictive method for assessing the probability of the success of embryo implantation based on methylation profiles of cervical secretions at the preimplantation stage.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows that a cervical sample is taken on day −5˜−1, and on the day of the embryo transfer, which is equivalent to the phase of day 1^{st}, 2^{nd}, 3^{st}, 4^{th}, or 5^{th} of progesterone administration in a hormone replacement therapy cycle (P+0˜P+5). FIG. 1B shows that the unsupervised hierarchical clustering is performed based on the methylome profiles with the top 2000 DMPs of cervical secretions across 16 samples (subject ID #344 day 0 & day 5, subject ID #342 day 0 & day 5, subject ID #350 day 0 and day 5, subject ID #107 day 0 and day 5, subject ID #314 day 0 and day 5, subject ID #239 day 0 and day 5, subject ID #041 day 0 and day 5, subject ID #339 day 0 and day 5). The data showed that most pairs of samples (same subject ID) have be assigned to the same cluster. Seven-eighths (⅞) paired clinical samples (subject ID #344, #342, #350, #107, #314, #239 and #339) showed similar methylation profiles at day 0 and day 5. P+0: start of progesterone intake.

FIG. 2A shows the scatter plot showing high correlations (R²=0.990) between repeated microarrays on the same sample. Each dot represents the β-value of a CpG site. FIG. 2B shows the volcano plot of differentially methylated probes (DMPs) between pregnancy and non-pregnancy groups. Each dot represents the differential methylation level of a CpG site, which is the median β-value in the non-pregnancy group minus that in the pregnancy group. Red and green dots represent significantly (P<0.05) hypermethylated (H) and hypomethylated (L) DMPs, respectively. NP: the non-pregnancy group. P: the pregnancy group.

FIG. 3A shows the five clusters resulted from k-means clustering. Two clusters (green and olive dots) comprised exclusively non-pregnancy samples and another two clusters (blue and orange triangles) comprised exclusively pregnancy samples. The last cluster comprised 9 pregnancy (pink triangle) and 6 non-pregnancy (pink dots) samples. FIG. 3B shows the t-distributed stochastic neighbor embedding (t-SNE) resulted in two clusters compatible with pregnancy status. NP: the non-pregnancy samples. P: the pregnancy samples.

FIG. 4A shows the unsupervised hierarchical clustering analysis of the 57 samples and the top 2000 DMPs. Samples are presented vertically and values of DNA methylation horizontally. The cyan and magenta columns represent pregnancy and non-pregnancy samples, respectively. The first cluster (C1) includes 3 pregnancy samples, the second cluster (C2) includes 24 pregnancy and 7 non-pregnancy samples, and the third cluster (C3) includes 1 pregnancy and 22 non-pregnancy samples. Other clinical parameters of the samples are listed as follows: the exposure to supraphysiological hormone levels caused by controlled ovarian hyperstimulation (COH), the presence of endometriosis, and the age of women receiving embryo transfer. FIG. 4B. On the left side of the heatmap the 2000 DMPs are clustered into three main clusters with differing characteristics by using hierarchical clustering, named cluster A, cluster B and cluster C. Indigo color represents highly methylated probes and yellow color unmethylated probes.

FIG. 5 shows the temporal transcriptome dynamics of selected genes in endometrial epithelial cells and stromal fibroblasts throughout the menstrual cycle. Data are retrieved from a publicly available single-cell RNA-seq (scRNA-seq) database. FSH: Follicle-stimulating hormone. LH: Luteinizing hormone.

### DETAILED DESCRIPTION OF THE INVENTION

The term “a” or “an” as used herein is to describe elements and ingredients of the present invention. The term is used only for convenience and providing the basic concepts of the present invention. Furthermore, the description should be understood as comprising one or at least one, and unless otherwise explicitly indicated by the context, singular terms include pluralities and plural terms include the singular. When used in conjunction with the word “comprising” in a claim, the term “a” or “an” may mean one or more than one.

The term “or” as used herein may mean “and/or.”

The endometrium is the mucosa coating the inside of the uterine cavity. Its function is to house the embryo, allowing its implantation and favoring the development of the placenta. This process requires a receptive endometrium capable of responding to the signals of the blastocyst, which is the stage of development of the embryo when it implants. Human endometrium is a tissue cyclically regulated by hormones, the hormones preparing it to reach said receptivity state are estradiol, which induces cell proliferation, and progesterone which is involved in differentiation, causing a large number of changes in the gene expression profile of the endometrium, which reaches a receptive phenotype for a short time period referred to as “window of implantation”. Therefore, the endometrial receptivity is the state in which the endometrium is prepared for embryo implantation. The present invention first demonstrates that gene methylation patterns from the cervical sample is associated with the change of endometrial receptivity during the pregnancy cycles.

The present invention provides a method for identifying a potential biomarker for determining the probability of the success of embryo implantation, comprising: (1) providing a cervical sample from a female subject; (2) assaying nucleic acids of the cervical sample to generate a methylation profile comprising 1733 genes listed in Table 4; (3) calculating a statistical value of at least one gene from the 1733 genes in the methylation profile; and (4) identifying the at least one gene as a biomarker in the cervical sample for determining the probability of the success of embryo implantation when the statistical value of the at least one gene is higher than a threshold value.

In one embodiment, the cervical sample is a biological sample obtained from the lumen of the cervix. The cervix is the lower part of the uterus in the human female reproductive system, composed of two regions; the ectocervix and the endocervical canal. The cervix connects the vagina with the main body of the uterus, acting as a gateway between them. Anatomically and histologically, the cervix is distinct from the uterus, and hence the present invention considers it as a separate anatomical structure. In a preferred embodiment, the biological sample comprises secretions, epithelial cells, stromal cells, squamous cells, glandular cells, immune cells, vaginal fluids, vaginal microbiota, mucus molecules or water.

In another embodiment, the cervical sample is obtained by using a cotton applicator, a cotton wool ball, a cotton swab, or cotton balls. It can be gently rubbed against the cervix to obtain samples.

An embryo transfer is part of the process of IVF. In one embodiment, the cervical sample is obtained from 1-5 days before or on the day of the female subject receiving embryo transfer. In other words, the cervical sample is obtained on day −5˜−1, or on the day of the female subject receiving embryo transfer. In a preferred embodiment, the cervical sample is obtained on day P+0, P+1, P+2, P+3, P+4 or P+5. In a more preferred embodiment, the cervical sample is obtained on day P+0 or day P+5. P+0 means the day of starting progesterone supplementation (considered as P+0). P+5 means the following 5^{th} day of progesterone supplementation or administration (considered as P+5). Progesterone can be applied orally, vaginally, intramuscularly, or subcutaneously. Different protocols for initiation of progesterone supplementation are reported, ranging from before oocyte retrieval to 6 days after oocyte retrieval. In current IVF practice, day 3 cleavage-stage embryo transfer and day 5 blastocyst-stage embryo transfer is routine in many assisted reproductive technology centers. A day 3 embryo should therefore be transferred 2 days earlier. In a preferred embodiment, the biological sample is obtained before embryo transfer during IVF.

The term “subject” as used herein, refers to an animal including the human species. Accordingly, the term “subject” comprises any mammal, which may benefit from the method of the present invention. The term “mammal” refers to all members of the class Mammalia. In one embodiment, the subject is a human.

The term “methylation” as used herein, refers to the covalent attachment of a methyl group at the C5-position of cytosine within the CpG dinucleotides of the core promoter region of a gene. The term “methylation state” refers to the presence or absence of 5-methyl-cytosine (5-mCyt) at one or a plurality of CpG dinucleotides within a gene or nucleic acid sequence of interest. As used herein, the term “methylation level” refers to the amount of methylation in one or more copies of a gene or nucleic acid sequence of interest. The methylation level may be calculated as an absolute measure of methylation within the gene or nucleic acid sequence of interest. Also, a “relative methylation level” may be determined as the amount of methylated DNA, relative to the total amount DNA present or as the number of methylated copies of a gene or nucleic acid sequence of interest, relative to the total number of copies of the gene or nucleic acid sequence. Additionally, the “methylation level” can be determined as the percentage of methylated CpG sites within the DNA stretch of interest.

As used herein, the term “methylation profile” refers to a set of data to representing the methylation level of one or more target genes in a sample of interest. In one embodiment, the methylation profile is generated by bisulfite sequencing PCR (BSP), reduced representation bisulfite sequencing (RRBS), whole genome bisulfite sequencing (WGBS), methylated DNA immunoprecipitation sequencing (MeDIP), enzymatic methyl sequencing (EM-Seq), mass spectrometry method, methylation specific PCR, qPCR, PCR, sanger sequencing, next-generation sequencer, methylation chip, methylation chip array, ion torrent sequencer, real-time nanopore sequencing, smaller genomes sequencing, targeted regions sequencing, targeted amplicons sequencing, fiber optical particle plasmon resonance (FOPPR), or changes in transverse proton relaxation. In a preferred embodiment, the methylation profile is generated by Infinium methylation array, a tiling microarray or methylation specific PCR.

The present invention uses a computational predictor to perform a mathematical tool which uses a data matrix, in this case of the data generated with the methylation profile, and learns to distinguish classes, in this case two or more classes according to the different pregnancy profiles that are generated (pregnancy and non-pregnancy). The set of samples which trains the classifier to define the classes is referred to as training set. In other words, the methylation profile of these samples, measured with the endometrial receptivity, are used by the program to know which probes are the most informative and to distinguish between classes (different normal non-receptive and receptivity states). This training set will gradually grow as a larger number of samples are tested.

The classification is done by the bioinformatic program using different mathematical algorithms, there being many available. An algorithm is a well-defined, ordered and finite list of operations which allows solving a problem. A final state is reached through successive and well-defined steps given an initial state and an input, obtaining a solution. The classifier calculates the error committed by means of a process called cross-validation, which consists of leaving a subset of the samples of the training set of a known actual class out of the group for defining the classes, and then testing them with the generated model and seeing if it is right. This is done by making all the possible combinations. The efficacy of the classifier is calculated and prediction models are obtained which correctly classify all the samples of the training set. In other words, all the samples of the training set are classified by the predictor in the assigned actual class known by the inventors.

Depending on all the parameters relating to the computational predictor explained above, a prediction model is generated which classifies all the samples according to the assigned actual class. Therefore, the genes of the methylation profile in the cervical sample can be used for the positive identification of the endometrial receptivity.

Therefore, the present invention also provides a method for identifying a potential gene associated with the probability of the success of embryo implantation, comprising: (a) providing a cervical sample from a female subject; (b) extracting nucleic acids from the cervical sample; (c) assaying the nucleic acids to generate a methylation profile; (d) in a programmed computer, inputting the data comprising the methylation levels of genes from the methylation profile in the step (c) to a trained algorithm to identifying one or more genes in the cervical sample associated with the success of embryo implantation based on the relationship between the methylation levels of genes and the change of endometrial receptivity; and (e) electronically outputting a report that identifies the one or more genes in the cervical sample associated with the probability of the success of embryo implantation.

The present invention uses a statistical analysis to process the differential methylation detection of the methylation profile from the cervical sample, then selects 1733 genes with the best performance listed in Table 4. The present invention further uses hierarchical models to cluster 1733 genes into three clusters, i.e., cluster A, cluster B and cluster C. According to the level in DNA methylation, the cluster A is a group with lower methylation (<10%) comprising 319 genes, the cluster B is a group with middle methylation (20%˜55%) comprising 174 genes and the cluster C is a group with higher methylation (>55%) comprising 1240 genes. In one embodiment, the 1733 genes are divided into cluster A comprising 319 genes, cluster B comprising 174 genes and cluster C comprising 1240 genes, wherein the genes in the clusters A, B and C are listed in Table 4.

The present invention further identifies multi-gene panels can serve as epigenetic biomarker panel for determining the probability of the success of embryo implantation. Therefore, the present invention selects at least one gene from clusters A, B and/or C to validate. In one embodiment, the at least one gene is selected from the group consisting of the cluster A, the cluster B and the cluster C. For example, the present invention identifies four, five or six-gene based panel. The AUC reached 0.81 (>0.8) in 4-gene combination (SYNE1, KCNC2, SLITRK2 and PDE4C). In another embodiment, the AUC was 0.81 in the 5-gene combination (SYNE1, KCNC2, SLITRK2, PDE4C and TMEM62). In another embodiment, the AUC was 0.82 in the 5-gene combinations (SYNE1, KCNC2, SLITRK2, PDE4C and ARID3C; SYNE1, KCNC2, SLITRK2, PDE4C and CASR). In another embodiment, the AUC was 0.82 in the 6-gene combinations (SYNE1, KCNC2, SLITRK2, PDE4C, CASR and TMEM62). In another embodiment, the AUC was 0.83 in the 6-gene combinations (SYNE1, KCNC2, SLITRK2, PDE4C, CASR and ARID3C).

The present invention also provides a composition comprising a gene combination, wherein the gene combination comprises SYNE1, KCNC2, SLITRK2 and PDE4C, and the gene combination is used for determining the probability of the success of embryo implantation. Therefore, the composition comprises multi-gene panels which can be used for determining the probability of the success of embryo implantation. More generally, the present invention identifies and validates multi-gene panels, that can predict clinical pregnancy outcome in IVF cycles with high precision.

In one embodiment, the gene combination further comprises at least one gene is selected from the group consisting of TMEM62, ARID3C and CASR.

The present invention uses a statistical method to calculate the statistical value of a gene panel from 1733 genes. Therefore, the present invention uses 5-fold cross-validation to assess classifier performance. The present invention applies 5-fold cross validation with 10 repetitions (500 iterations) for each of the datasets. The maximum and minimum AUCs are calculated (over the 500 iterations). The AUC is averaged from all 500 repetitions of bootstrap sampling, and the confidence intervals are computed from the concatenation of the predicted and actual values through these iterations. In one embodiment, the statistical value of the at least one gene is a value of an area under the curve (AUC) calculated by a receiver operating characteristic (ROC) curve. In a preferred embodiment, the statistical value of the at least one gene is a value of AUC calculated by k-fold cross validation, wherein the k is an integer. In another embodiment, the k is 4, 5, 10, 20, 50, 100 or 500. In a preferred embodiment, the k is 5 or 500. In one embodiment, the value of AUC is calculated by k-fold cross validation which is performed based on the methylation profile of the cervical samples from the non-pregnancy group after receiving embryo implantation and the pregnancy group after receiving embryo implantation, wherein the k is an integer. In a preferred embodiment, the value of AUC is calculated by 500-times bootstrapping which is performed based on the methylation profile of the cervical sample from the non-pregnancy group after receiving embryo implantation and the pregnancy group after receiving embryo implantation.

In addition, the threshold value is determined by a data point on the ROC curve. In one embodiment, the threshold value is 0.5. In a preferred embodiment, the threshold value is 0.7. In a more preferred embodiment, the threshold value is 0.8. In another embodiment, the threshold value is 0.9.

The present invention further provides a kit for determining the probability of the success of embryo implantation, comprising a composition, wherein the composition comprises first binding molecules for detecting SYNE1, KCNC2, SLITRK2 and PDE4C.

In one embodiment, the composition further comprises second binding molecules for detecting at least one gene, wherein the at least one gene is selected from the group consisting of TMEM62, ARID3C and CASR.

In one embodiment, the form of the binding molecules comprises antibodies, peptides, primers or probes.

The present invention reveals that DNA methylation profiles from cervical secretions differed between pregnancy and non-pregnancy cycles. Using cervical secretions obtained during procedures of embryo transfer, the accuracy of using the methylation status for predicting pregnancy outcomes can be as high as 86.0%, providing a new way to personalize embryo transfer.

The advantage of the present invention is the use of a noninvasive approach that enables confirmation of the test results using pregnancy outcomes. The detection of the cervical secretions for analyzing is able to ensure the avoidance of perturbation of the implantation environment, providing a tool to investigate the monthly variation of endometrial receptivity. Because the analyzed cycle is the conceptional cycle itself, this noninvasive analysis is applicable to both fresh and frozen-thawed embryos. Even for in vivo fertilized embryos in natural conception, this noninvasive test is a promising way of indicating fertile cycles by identifying the receptive endometrium.

Thus, the present invention demonstrates the feasibility of noninvasively assessing endometrial receptivity using methylation status as determined from cervical secretions. The methylation profiles of mid-secretory samples can identify 96.4% of receptive endometria, as confirmed by a viable ongoing pregnancy after embryo transfer in the very same cycle. Predicting receptivity of the endometrium ahead of embryo transfer through quick diagnostic tests can maximize the likelihood of a successful pregnancy by saving good embryos for cycles with a favorable endometrium. The methylation profile not only provides an objective diagnosis of endometrial receptivity, but also reveals the molecules involved in the establishment of pregnancy, which may pave the way for new therapies for endometrial and obstetric diseases.

### Examples

The examples below are non-limiting and are merely representative of various aspects and features of the present invention.

Materials and Methods:

1. Clinical Samples

The samples were collected from 2018 to 2021. Cycles with at least one good quality embryo ready for transfer were included in this present invention. Written informed consent was obtained with the approval of the ethic committee from all participating women. Embryos of good quality were defined as follows: (1) cleavage-stage embryos with an adequate number of cells (4-5 cells on day 2 and 7-9 cells on day 3 of culture) as well as less than 20% fragmentation and (2) blastocysts scored ≥3BB according to the Gardner and Schoolcraft grading system.

A sample of cervical secretion was collected during the embryo transfer procedure. The samples used in the present invention were obtained from a cervical sample before embryo transfer. The cervical sample from the female subject should be taken on the 0, 1^{st}, 2^{nd}, 3^{st}, 4^{th}, or 5^{th} day of progesterone administration (P+0˜P+5) in a hormone replacement treatment (HRT) cycle (with progesterone administration), or in a natural cycle controlled by human chorionic gonadotropin (hCG) with and without modifications for ovulation triggering. In the case, embryo transfer should be carried out on the 5^{th}, 6^{th} or 7^{th} days after oocyte retrieval. Samples were categorized into the pregnancy group and the non-pregnancy group according to the existence of a viable intrauterine pregnancy at 12 weeks of gestation. Overall, 59 pregnancy and 67 non-pregnancy samples were used for matched analysis. These samples were separated into a discovery set and a validation set. The methylomic profiles were generated using the discovery set, including 27 pregnancy and 30 non-pregnancy samples, which were subsequently used for verification of the array data. The validation set included 32 pregnancy and 37 non-pregnancy samples, which were used to validate the methylation levels of the selected genes (Table 1). Clinical characteristics of the enrolled embryo transfer cycles were recorded, including the age of the women at embryo transfer, the presence of endometriosis, the use of ovarian stimulation, and the number of embryos per transfer. Fresh embryos were transferred after IVF following ovarian stimulation and oocyte retrieval. In cycles of frozen embryo transfer, the endometrium was prepared by hormone replacement treatment. For women with endometriosis or adenomyosis, the preparation of endometrium was preceded by pituitary downregulation for at least 1 month.

2. DNA Extraction

The cervical secretions were collected before embryo transfer procedure (P+0˜P+5) using a cotton wool ball, and put into a 50 ml centrifuge tube, and stored at 4° C. One milliliter of phosphate buffered saline was used to rinse the cotton wool ball, which was then centrifuged at 1000 g for 10 min to collect the flow-through. Genomic DNA was extracted from the flow-through using the QIAamp DNA Mini Kit (QIAGEN, Hilden, Germany). DNA extracts were stored at −20° C. or −80° C. before use.

3. Differential Methylomics and Bioinformatic Analysis

The present invention generated methylomic profiles of samples from the discovery set using the Infinium MethylationEPIC BeadChip array, which covered more than 850,000 CpG sites (Illumina, San Diego, Calif., USA). In the beadchip system, β-value (ranging from 0 to 1), where 0.0 is equivalent to 0% methylation and 1.0 is equivalent to 100% methylation at a given CpG dinucleotide, was used to present the DNA methylation level of each probe. The methylation levels derived from type I and type II probes were normalized by the Beta-Mixture Quantile (BMIQ) method. After probes with single-nucleotide polymorphism (SNP) were removed, the differentially methylated probes (DMPs) were identified by a detecting P value of each probe <0.05 and a β difference>|0.02|. Next, the present invention focused on DMPs at promoter regions and ranked them by the area under the receiver operating characteristic curve (AUC). A higher AUC meant higher accuracy in differentiating pregnancy and non-pregnancy samples. The performance of various DMP sets, such as Top 3000, Top 2000, and Top 1500, was evaluated by the percentage of correct categorization of samples in terms of pregnancy outcomes. The top 2000 DMP set had the best performance and was selected for the following analysis (Table 2).

4. Bisulfite Conversion

DNA was bisulfite-converted from 500 pg-2 μg genomic DNA, cDNA or fragmented DNA, using the EZ DNA Methylation Kit, EZ DNA Methylation-Direct Kit, EZ DNA Methylation-Gold Kit, EZ DNA Methylation-Lightning Kit (Zymo Research Corp., Irvine, Calif., USA) or other commercial kits, according to the manufacturer's recommendations.

5. Statistical Analysis

The Mann-Whitney nonparametric U test was used to identify differences in methylation levels between the two sample groups. The significance of all differences was assessed using a two-tailed t test for continuous variables and Fisher's exact test for categorical variables, with a threshold for significance of P<0.05. AUC was calculated using the Youden index in the ROC package. To estimate the performance of gene combinations in predicting pregnancy outcomes, a logistic regression model based on 500 rounds of five-fold cross-validation on all samples was performed to calculate AUC. The aforementioned analyses were performed and the plots were created using the statistical package in R (version 3.3.2) or MedCalc version 19 (MedCalc Software Ltd., Ostend, Belgium; 2018).

6. Biomarker Panel Selection

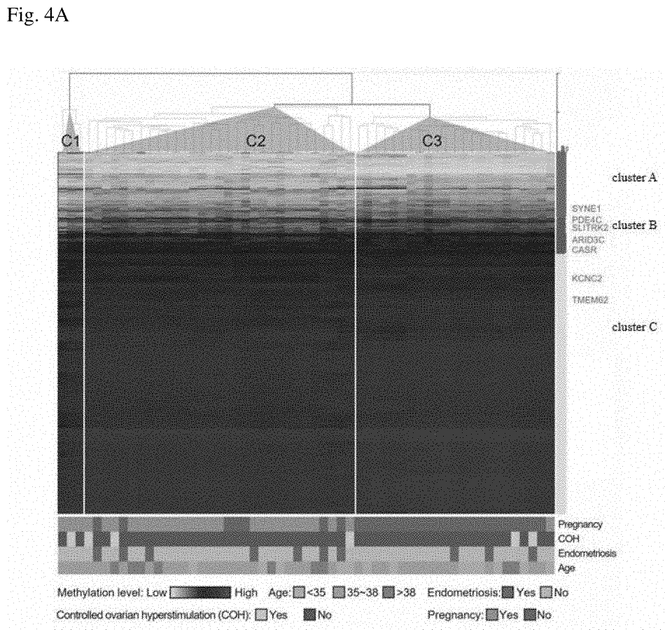

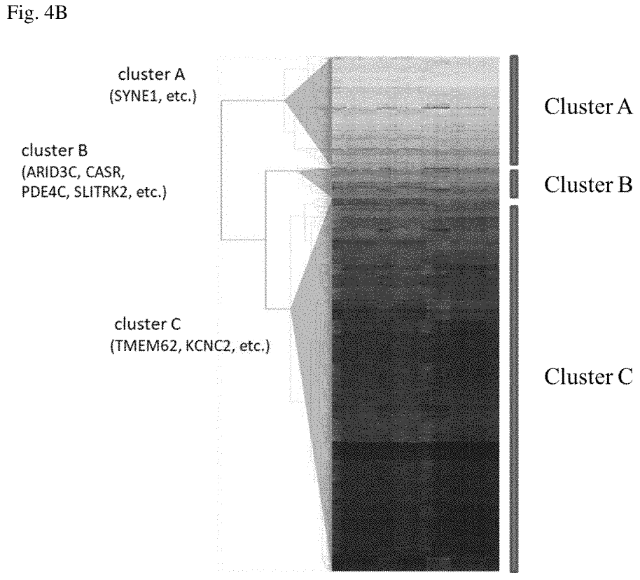

Heat map analysis combined with hierarchical clustering was performed to investigate whether the 2000 DMPs clearly differentiated between the non-pregnancy group and the pregnancy group (FIG. 4A). FIG. 4B shows the unsupervised hierarchical clustering analysis of the top 2000 DMPs.

7. Measurement of Methylation Levels by qMSP

To verify the array data, a biomarker panel is designed and validated. One to two genes in each subgroup of the top 2000 DMPs were selected for quantifying DNA methylation levels using real-time polymerase chain reaction. The primers were designed by Oligo 7.0 Primer Analysis software (Molecular Biology Insights, Inc., Colorado Springs, Colo., USA). Quantitative methylation-specific polymerase chain reaction (qMSP) assays were performed on the LightCycler 480 System (Roche, Indianapolis, Ind., USA). Duplicate testing was conducted for each gene in all samples. To normalize the amount of input DNA in each qMSP reaction, a type II collagen gene (COL2A1), located in a non-CpG region, was used as a reference. DNA methylation levels were estimated by the difference in crossing point (ΔCp) values, defined as follows: Cp of target gene-Cp of COL2A1. Samples with test results of a Cp value of COL2A1>36 were defined as not detectable.

8. Hierarchical Cluster Analysis

Hierarchical cluster analysis is a step-by-step process to perform a cluster analysis. Calculated the distance matrix by Euclidean or Manhattan distance and complete linkage method to generate a dendritic tree. Using the distance threshold separates optimal subgroups.

Results:

1. Genome-Wide Methylation Profiles of Cervical Secretions

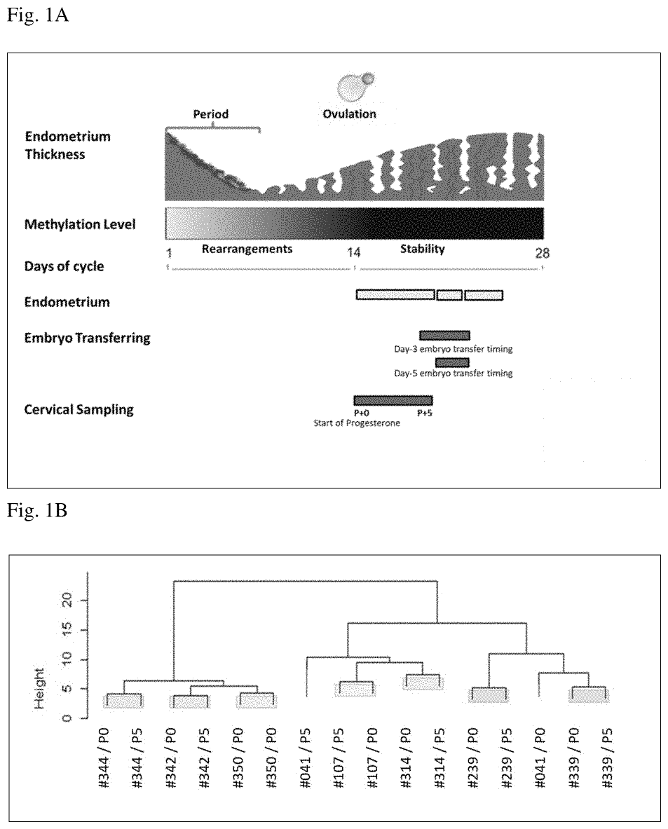

As illustrated in FIG. 1A, the present invention measured the genome-wide DNA methylation profiles of cervical secretions that were collected before embryo transfer using the Infinium MethylationEPIC BeadChip array (Illumina, San Diego, Calif., USA). The cervical samples used in the present invention were obtained before embryo transfer. The present invention revealed DNA methylation profiles from cervical secretions were different between pregnancy and non-pregnancy cycles. Using cervical secretions obtained during procedures of embryo transfer, the endometrial receptivity can be assessed. As illustrated in FIG. 1B, the methylation profiles of cervical secretions on day 0 (P+0) and day 5 (P+5) were relatively similar.

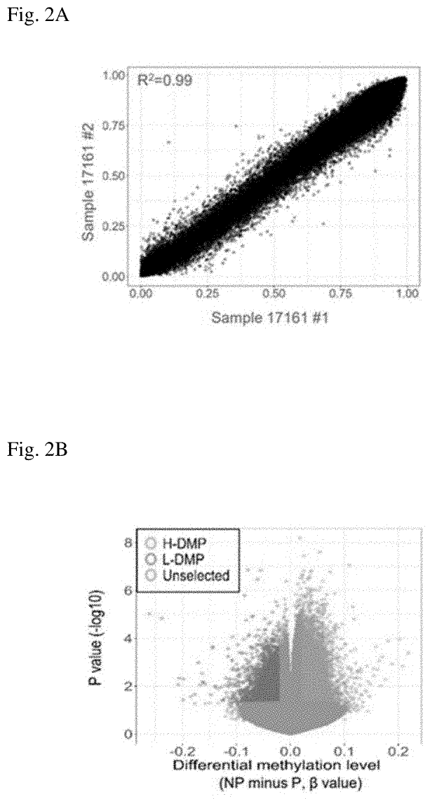

Samples of cervical secretions were categorized as pregnancy and non-pregnancy according to the existence of a viable intrauterine pregnancy at 12 weeks of gestation following embryo transfer. The discovery set included 28 pregnancy and 29 non-pregnancy samples. Clinical characteristics of embryo transfer cycles enrolled in the discovery set are described in Table 1. The measurement of methylation levels was reliable, as shown by the high correlation (R²=0.99) between technical replicates (FIG. 2A). Of the 739,266 probes remaining after quality control filtering, after normalization, the methylation profiles of cervical secretions from pregnancy and non-pregnancy cycles were relatively similar.

TABLE 1
Clinical characteristics of samples

| | Discovery set | | | Validation set | | |
|---|---|---|---|---|---|---|
| | | Non- | | | Non- | |
| Clinical | Pregnancy | pregnancy | | Pregnancy | pregnancy | |
| characteristics | (n = 28) | (n = 29) | P value | (n = 32) | (n = 37) | P value |
| Age (years) | 36.3 ± 2.7 | 35.8 ± 2.1 | 0.64 | 37.3 ± 5.0 | 40.7 ± 4.4 | <0.01 |
| Presence of | 3 (10.7) | 7 (24.1) | 0.30 | 7 (21.9) | 9 (24.3) | 1.00 |
| endometriosis | | | | | | |
| Presence of ovarian | 4 (14.3) | 2 (6.9) | 0.42 | 2 (6.3) | 3 (8.1) | 1.00 |
| stimulation | | | | | | |
| Number of embryo | 2.2 ± 0.7 | 2.3 ± 0.7 | 0.79 | 2.2 ± 0.8 | 2.2 ± 1.0 | 1.00 |
| per transfer (n) | | | | | | |

Data are mean ± standard deviation or n (%). P values were calculated by t test or Fisher's exact test. n: number.

There were 23569 CpG sites with significant differences in methylation between pregnancy and non-pregnancy samples, accounting for 3.2% of total probes (FIG. 2B). With regard to genomic locations, the majority of differentially methylated probes (DMPs) were located in gene body regions, followed by intergenic regions. In relation to CpG islands, most DMPs were concentrated in open sea.

2. Predicting Pregnancy Outcomes by Differential DNA Methylation

Unsupervised hierarchical clustering analysis of all DMPs correctly categorized 45 out of the 57 samples (78.9%) according to pregnancy status (Table 2). The percentage of correct categorization became higher (84.2%) when only the 5569 DMPs located at promoter regions were used for analysis (Table 2). The present invention further eliminated less relevant probes to identify the panel with the best performance by ranking the promoter DMPs according to AUC, which represented the ability of methylation levels to separate pregnancy from non-pregnancy samples. During this process, the percentages of correct categorization for all samples as well as for pregnancy samples increased until the size of DMPs was less than 2000 (Table 2). The top 2000 promoter DMPs were 86.0% correct for all samples and 96.4% correct for pregnancy samples, which constituted the profile with the fewest probes and the best performance for differentiating pregnancy and non-pregnancy samples.

TABLE 2
Performance of differential DNA methylation for
predicting pregnancy outcomes

| | | Correct categorization, n (%) | | |
|---|---|---|---|---|
| | | Pregnancy | Non-pregnancy | |
| | | samples | samples | All samples |
| DMP sets | Threshold | (n = 28) | (n = 29) | (n = 57) |
| All DMPs | 1823.0 | 19 (67.9) | 26 (89.7) | 45 (78.9) |
| Promoter DMPs | 438.1 | 22 (78.6) | 26 (89.7) | 48 (84.2) |
| Top 3000 | 146.8 | 27 (96.4) | 22 (75.9) | 49 (86.0) |
| promoter DMPs | | | | |
| Top 2000 | 150.3 | 27 (96.4) | 22 (75.9) | 49 (86.0) |
| promoter DMPs | | | | |
| Top 1500 | 112.2 | 21 (75.0) | 25 (86.2) | 46 (80.7) |
| promoter DMPs | | | | |

The results were calculated by unsupervised hierarchical clustering with Manhatten distance and complete linkage. DMP: differential methylated probe. n: number.

Analysis of the top 2000 DMPs by unsupervised hierarchical clustering was performed, as shown in Table 3, which revealed three main clusters that divided the 57 cervical secretion samples according to pregnancy outcomes. The first cluster (C1) included 3 samples all from pregnancy cycles. The second cluster (C2) included most of the pregnancy samples, that is, 24 pregnancy and 7 non-pregnancy samples. In contrast, most of the non-pregnancy samples clustered in the third cluster (C3), which included 22 non-pregnancy samples and only one pregnancy sample (Table 3). Factors that may influence pregnancy outcomes were analyzed, such as the age of women receiving embryo transfer, the presence of endometriosis, and the exposure to supraphysiological hormone levels due to ovarian stimulation. None of the above factors was correlated with the three clusters, implying the specificity of the selected DMPs to pregnancy status.

TABLE 3
The hierarchical cluster analysis in 57 cervical
samples, 27 of pregnancy (P group) and 30 of non-pregnancy (nP group).

| Classifier groups | C1 | C2 | C3 |
|---|---|---|---|
| | (High CPR) | (Middle CPR) | (Low CPR) |
| Clinical pregnancy rate | 3/3 | 24/31 | 1/23 |
| (CPR) | (100%) | (77.4%) | (4.3%) |

Clinical pregnancy is defined as the presence of an intrauterine gestational sac under ultrasound scanning 5 to 6 weeks after the embryo transfer.

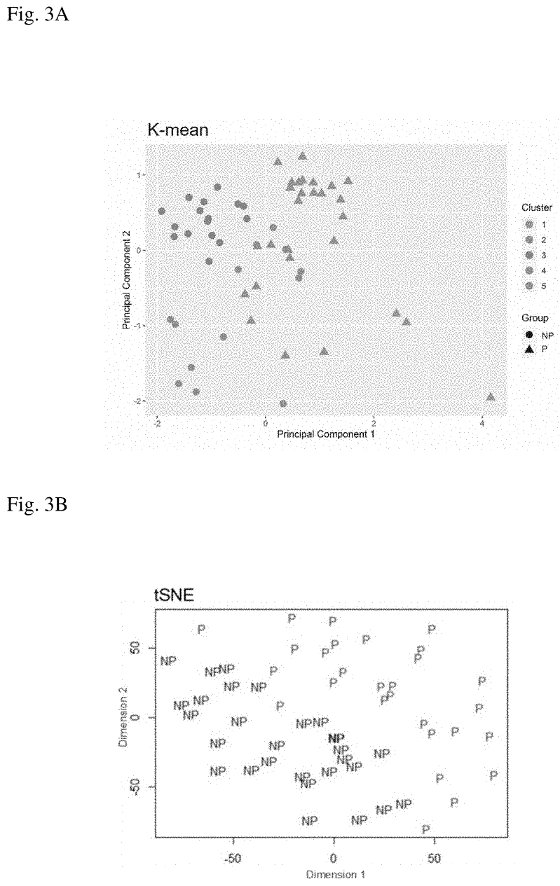

The ability of the top 2000 DMPs to classify samples according to pregnancy outcomes could also be characterized with other machine learning techniques. Upon analysis by k-means clustering, the top 2000 DMPs partitioned the 57 samples into 5 clusters. Two clusters comprised exclusively pregnancy samples and another two clusters comprised exclusively non-pregnancy samples. There was only one cluster comprising both samples, which included 15 samples from 9 cases of pregnancy and 6 of non-pregnancy (FIG. 3A). The present invention used t-distributed stochastic neighbor embedding (t-SNE), a nonlinear dimensionality reduction technique, to visualize the top 2000 DMPs in two-dimensional space, which categorized the 57 samples into two clusters compatible with pregnancy status (FIG. 3B). Accordingly, DNA methylation profiles in cervical secretions were capable of differentiating pregnancy cycles from non-pregnancy cycles, suggesting how methylation status reflects endometrial receptivity.

3. Microarray Verification by qMSP

To verify how methylation status reflects pregnancy status as discovered by microarray, the methylation levels of selected genes were measured by qMSP using the same samples from which the microarray results were generated. The genes associated with top 2000 DMPs included 1733 genes. Table 4 showed the 1733 candidate genes. Simultaneously, the present invention minimized the number of features to select the best multi-biomarker panel for pregnancy outcome prediction. The 1733 genes candidate genes could be divided into 3 clusters, A, B and C (Table 4). The algorithm also clustered top 2000 DMSs three major groups consisting of 355 DMSs in cluster A (comparatively hypomethylated); 191 DMPs in cluster B; and 1454 DMPs in cluster C (comparatively hypermethylated).

TABLE 4
List of 1733 candidate genes of top 2000 DMPs
top 2000 DMPs (1733 genes)

| | |
|---|---|
| cluster A | ABL1, ACACA, ACN9, ACSM3, ACTA1, ACTN1, ACVR1C, |
| (319 genes; 355 DMPs) | ADAMTS16, ADGRG2, AEN, AIFM1, AIG1, AMPH, ANK2, ANKRD11, |
| | ANP32E, ARHGEF6, ARMCX3, ARMCX5, ARMCX5-GPRASP2, |
| | ATP12A, ATP2C1, ATRX, AURKB, AZIN1, BACH2, BARHL1, BCAM, |
| | BCAR1, BCAT1, BDNF, BDP1, BEX5, BLOC1S1, BRAF, C10orf105, |
| | C10orf26, C12orf75, C19orf41, C3orf67-AS1, C8orf48, C8orf85, C9orf109, |
| | CA14, CACNA1S, CARTPT, CASC11, CCDC33, CD48, CDH12, CENPB, |
| | CENPV, CFLAR, CHAF1B, CHODL, CHRNB2, CHST12, COL13A1, |
| | COL4A5, COL4A6, COPS7A, CPNE6, CRADD, CREB5, CSTF2, |
| | CTAGE5, CTHRC1, CTNND1, CTNND2, CXCL1, CXCL6, CYB5A, |
| | CYBA, DBT, DCAF12L2, DCLK1, DEDD, DEMI, DERL2, DGKQ, |
| | DMRTA1, DNAJC14, DOCK9-AS2, DPH1, DZIP1, ERCC6L, ESR1, |
| | FADS2, FAM122C, FAM123B, FAM20B, FAM3A, FAM47E-STBD1, |
| | FAM96A, FANCB, FAT1, FBN2, FBXO22, FGF5, FRY-AS1, FUNDC2, |
| | FZD5, GABRA1, GALNT13, GDNF, GEFT, GHSR, GLRA3, GMNN, |
| | GPC3, GPC4, GSN, GUCY1A2, HDAC5, HDGF2, HDX, HES6, HIVEP2, |
| | HMGN5, HMX3, HOXD3, HOXD4, HS3ST3A1, IDH3G, IFITM1, |
| | IL1RAPL2, ILK, IQGAP1, IRX4, ISLR2, ITGB3BP, KCNH5, KCTD18, |
| | KDM4B, KIRREL3, KPNA2, L1TD1, LAS1L, LIN54, LMO3, |
| | LOC100128731, LOC101927322, LOC283999, LOC285830, LRCOL1, |
| | LRFN2, LRRC4B, LRRFIP2, LZTS2, MAP7D2, MAP7D3, MBNL2, |
| | MDM4, MECOM, MEF2C, MFI2, MLC1, MSC, MT1G, MTIF3, |
| | MUPCDH, MYL6B, MYOD1, NAF1, NAT15, NDUFA12, NEILl, NEMF, |
| | NEU1, NFATC4, NFKBIZ, NHS, NHSL1, NKAIN3, NKX2-4, NLGN1, |
| | NPY2R, NRK, NXPH1, OCRL, OLIG2, OTUB1, OTUD3, OTX2OS1, |
| | OXA1L, P3H4, PAQR9, PAX3, PCDHB1, PCDHGB1, PCGF3, PCK2, |
| | PCOLCE2, PHC1, PHC2, PHF8, PKNOX2, PLEKHF2, PLK4, PLS3, |
| | PMPCB, PNPLA5, POT1-AS1, PPFIA2, PPP1R3B, PPP2CB, PRDM2, |
| | PRICKLE1, ProSAPiP1, PRR36, PTCHD1, PTPRD, RAB39B, RASL11B, |
| | RBM12B-AS1, RBM15B, RBM20, RBM3, RBM41, RBP4, RBP7, |
| | REPIN1, RERE, RFC3, RFX8, RPL22L1, RPL39, RPS3, RRAGB, |
| | RUVBL1, SCML2, SERTAD4, SF3B5, SFTA3, SHANK3, SIX2, SKOR1, |
| | SLC16A7, SLC1A3, SLC22A16, SLC25A14, SLC2A14, SLC33A1, |
| | SLC35A2, SLC5A6, SLC7A10, SLN, SNORD42B, SNORD50B, SNTB1, |
| | SNTG1, SNX14, SOX1, SP110, SPATS1, SPG11, SPIN2B, SRC, |
| | ST6GAL1, STAU2, STK39, SYNE1, TAC1, TCEAL1, TCN2, THRAP3, |
| | THY1, TMEM131, TMEM187, TMEM196, TMEM219, TMEM246, |
| | TMSL3, TNFAIP8, TNFAIP8L2, TP53RK, TRIM26, TRIM68, TSC22D3, |
| | TSSC4, TTC23, TTLL4, TUBB4, TUBB4A, TUBE1, UBA1, UBXN10, |
| | UNC13B, USP28, USP37, VGF, VMP1, VWDE, WAC, WASF3, WNT16, |
| | WNT4, WSCD1, WWC1, WWC3, ZC3H12A, ZC4H2, ZDHHC22, ZFR2, |
| | ZIC3, ZIC4, ZIC5, ZNF212, ZNF335, ZNF397OS, ZNF449, ZNF490, |
| | ZNF560, ZNF562, ZNF662, ZNF75A, ZNF891, ZNF98, ZNRF1 |
| cluster B | ACY3, ADCYAP1R1, AMN1, AMZ1, ANKRD12, ANKRD33, APOOL, |
| (174 genes; 191 DMPs) | AQR, AR, ARID3C, ARMCX4, ASCL2, ATP8B4, BCOR, BTNL9, BUB3, |
| | C10orf126, CACNG6, CALCR, CASR, CBFA2T3, CCDC52, CCDC92, |
| | CD209, CDH22, CDHR2, CHRM4, CHST7, CLEC4GP1, CNTNAP4, |
| | COLEC11, DAD1, DIRAS3, DLC1, DNTT, DOCK11, DUSP14, EGFL6, |
| | ELF4, ELF5, ELK1, ELOVL2-AS1, ERAS, ERMN, FAM135A, FGF13, |
| | FILIP1, FLJ40504, G3BP1, GABRG3, GABRQ, GALR1, GJD3, GNE, |
| | GNG13, GNL3L, GORAB, GPR82, GRIA2, GRIN2D, GTF3C5, H19, |
| | HECW1, HMGB3, HOXA5, HTATSF1, IKBKG, INO80C, INPP4A, IRF5, |
| | KCND1, KCNQ1OT1, KCTD16, KDM6B, KRT40, KRTAP2-3, L3MBTL, |
| | LIFR, LIMK1, LINC01056, LINGO3, LOC102724050, LOC285370, |
| | LOC401010, LOC729176, LOC90110, LRP2BP, LRRN4, LVRN, |
| | LYSMD4, MADD, MAP3K15, MATR3, MCART6, MFSD4, |
| | MGC57346-CRHR1, MIR663A, MIR886, MLIP-IT1, MMAA, MRPL24, |
| | MUC12, MYF5, NHSL2, NOS3, NREP, NRIP1, NRXN3, NTN4, |
| | NUP62CL, PACSIN1, PAPOLA, PCBP3, PCDHB14, PCDHB3, PDE4C, |
| | PEG3, PIM2, PLAGL1, PLCH2, POU2F2, PPP1R9A, PPP6R3, PRKCZ, |
| | PROC, PRR23C, PRSS50, PSMA6, RAB27B, RAP2C, RBFOX1, RNF219, |
| | RPL36, S100A16, SCML1, SEPT9, SH3BP2, SLC10A4, SLC23A1, |
| | SLC24A5, SLC26A9, SLC35C1, SLC7A3, SLITRK2, SMTNL2, SNCA, |
| | SNRPN, SOX3, SPAG1, SPAG4, SPATA13, SPATA20, STAG2, STARD8, |
| | SYP, TMEM168, TMEM220, TREM1, TRIM21, TSLP, TTC33, USP29, |
| | USP51, VBP1, WDR19, WDR45, WDR88, YTHDF2, YY2, ZFHX2, |
| | ZFYVE27, ZNF239, ZNF319, ZNF75D |
| cluster C | AADACL4, AASS, ABAT, ABCA10, ABCA5, ABCA9, ABCB5, ABCC10, |
| (1240 genes; 1454 DMPs) | ABHD2, ABI3BP, ABTB1, ACSL1, ACTR3C, ACVR1, ADAL, ADAM2, |
| | ADAM20, ADAM30, ADAMTS2, ADARB1, ADCY7, ADD2, ADH1B, |
| | ADIPOR2, ADSSL1, AFF3, AGBL2, AGK, AGPAT1, AIFM2, AKAP11, |
| | AKAP13, AKAP6, ALAD, ALKBH3, ALLC, AMTN, ANGPT2, |
| | ANGPTL3, ANK3, ANKIB1, ANKRD28, ANKRD30B, ANKRD49, |
| | ANKRD55, ANP32C, ANXA9, APBB1IP, APCDD1, APP, ARHGAP12, |
| | ARHGAP15, ARHGDIB, ARHGEF18, ARHGEF28, ARHGEF38-IT1, |
| | ARHGEF4, ARL14EP, ARMC1, ARMC3, ARMC4, ARPP19, ARPP21, |
| | ARSE, ART1, ASB11, ASB15, ASB5, ASS1, ASZ1, ATP10A, ATP10B, |
| | ATP13A5, ATP13A5-AS1, ATP4B, ATP5SL, ATP6V0A4, AVP, AZI2, |
| | B3GALT1, B3GALT4, B3GALT5, B3GAT1, BAAT, BANP, BDKRB1, |
| | BHMT, BIRC8, BIVM, BLID, BMI1, BMPR1A, BMX, BOLL, BRDT, |
| | BTNL3, C10orf113, C10orf140, C10orf47, C11orf39, C12orf35, C12orf42, |
| | C12orf43, C12orf69, C13orf1, C13orf33, C13orf39, C14orf180, C14orf184, |
| | C15orf51, C15orf53, C15orf54, C15orf60, C16orf5, C1GALT1, C1orf122, |
| | C1orf234, C1orf61, C1orf87, C1QL2, C1QTNF7, C1QTNF8, C21orf58, |
| | C21orf90, C2orf63, C2orf80, C2orf86, C2orf88, C3orf57, C5orf36, |
| | C5orf43, C5orf47, C5orf66, C6, C6orf201, C7, C7orf16, C7orf27, C7orf62, |
| | C7orf65, C7orf66, C8A, C8B, C8orf44-SGK3, C9orf129, C9orf135, |
| | C9orf57, CA3-AS1, CAB39L, CABC1, CACNA1F, CACNA2D2, |
| | CACNG7, CADM2, CALCB, CALCRL, CALN1, CALU, CARD8, |
| | CASP10, CATSPER4, CBX7, CCDC178, CCDC23, CCDC25, CCDC82, |
| | CCL20, CCNB1IP1, CCNY, CCR2, CCSER1, CD160, CD1D, CD200, |
| | CD226, CD2AP, CD37, CD79B, CDC16, CDH18, CDH19, CDK14, |
| | CDK17, CDK20, CDK3, CDKL5, CDRT7, CENPP, CES5A, CFAP126, |
| | CFAP43, CFAP44-AS1, CFHR4, CGA, CGB1, CHCHD7, CHD1L, CHD9, |
| | CHL1, CHN1, CHN2, CHRNA4, CHST2, CHST8, CHSY3, CIAPIN1, |
| | CLCC1, CLDN18, CLDN24, CLIP1, CLLU1OS, CLN5, CLRN1OS, |
| | CLVS1, CMBL, CNGA1, CNST, CNTN4, CNTN4-AS2, CNTN5, |
| | COL28A1, COL8A1, COL9A1, COLEC10, COMTD1, CORO1C, |
| | CORO2B, COX6A2, COX7B2, CPA2, CPAS, CPEB1, CPLX2, CRAT8, |
| | CRH, CRISP2, CRX, CRYGD, CSF1R, CSF2RB, CSNK1G1, CSNK1G2, |
| | CSRP3, CT45A1, CT47A1, CTAG2, CTB-12O2.1, CTBP2, CTNNA2, |
| | CTNNBIP1, CTSS, CTXN3, CWF19L2, CXCR1, CXCR6, CXorf61, |
| | CXXC1, CYP11B2, CYP19A1, DAAM1, DAB1, DAPK1, DCP1A, |
| | DCSTAMP, DCUN1D3, DDC, DDHD1, DDX53, DEF6, DEFB124, |
| | DENND4A, DGKH, DHX35, DISC1FP1, DKFZP586I1420, DKK3, DLD, |
| | DLG2, DLGAP4, DNAH5, DNAH9, DNAJB13, DNAJC6, DNASE2B, |
| | DOCK8, DPEP1, DPPA2, DPPA5, DPT, DRD3, DRD4, DROSHA, DSG4, |
| | DSPP, DST, DUXA, DYNC1I1, DYTN, EBAG9, EDNRB, EFCAB3, |
| | EFCAB5, EFCAB6-AS1, EIF1AX-AS1, EIF4E1B, EIF4G3, ELAVL1, |
| | ELF1, ELOVL5, ENOX2, ENTPD6, EPB41, EPB41L2, EPB41L3, EPOR, |
| | EPPK1, EPR1, EPS8, EPX, ERICH2, ESF1, ETF1, EXOSC9, EXT2, |
| | EYA2, FAM107B, FAM110B, FAM135B, FAM163A, FAM163B, |
| | FAM169B, FAM180A, FAM190A, FAM192A, FAM24A, FAM55D, |
| | FAM71F1, FAM9C, FANCA, FARS2, FASTK, FAT3, FBXL21, |
| | FBXO22OS, FBXO34, FBXW7, FCRL2, FDCSP, FER1L5, FER1L6, |
| | FGF12, FGF14-AS1, FGFR4, FGGY, FGR, FHIT, FLG2, FLJ41941, |
| | FLJ46361, FLRT3, FMN1, FOXL1, FOXN3, FOXP1, FPR2, FREM1, |
| | FRMD1, FRS3, FTMT, FTSJD1, FUOM, FUT8, FUT9, FXR1, FYN, |
| | G3BP2, GABRA4, GALM, GAP43, GAS2, GCC2, GCSAML, GCSH, |
| | GDF1, GDPD2, GEMIN7, GHR, GIGYF1, GJA8, GJA9, GK2, GK5, |
| | GLCE, GLIS1, GLIS3, GLYAT, GMFG, GNAL, GNRH1, GOLSYN, |
| | GOT1L1, GPCPD1, GPHB5, GPI, GPR1, GPR35, GPR44, GPRIN3, |
| | GPSM2, GRAMD1B, GRB10, GSG1, GTF2IRD1, GUCY1B3, GUCY2G, |
| | HABP2, HAO2, HBBP1, HCK, HDAC4, HDGFL1, HECTD4, HELLS, |
| | HESX1, HHATL, HHLA2, HILS1, HIST1H1T, HIST3H3, HLA-DQA2, |
| | HLCS, HMBOX1, HMCES, HMCN2, HNF1A, HOMER3, HOXA3, HRG, |
| | HRH1, HRNBP3, HSBP1L1, HSD11B1, HSD17B11, HSD17B4, HSDL1, |
| | HTR2A, HTR2A-AS1, HTR2B, HTR2C, HTR3C, HTR3D, HTRA3, |
| | ICA1L, IFNA8, IFNE, IGFBP1, IGFL1, IGFN1, IGSF11, IL15, IL17RD, |
| | IL1R1, IL21, IL24, IL5, INPP4B, INPP5D, INSIG2, INSL3, IPO5, IRF9, |
| | ITGA11, ITGB1BP3, ITGBL1, JAK1, JAM3, KCCAT198, KCNAB1, |
| | KCNC2, KCNIP1, KCNIP4, KCNJ16, KCNMB3, KCNS3, KCNU1, |
| | KCTD4, KDM4C, KDM6A, KDM8, KIAA0182, KIAA0513, KIAA0748, |
| | KIAA1024L, KIAA1191, KIAA1217, KIF1A, KIF2B, KIF4B, KIF6, KIF9, |
| | KIFC3, KIR3DL2, KLF14, KLF17, KLHDC7A, KLHL13, KLHL20, |
| | KLHL24, KLHL28, KLHL29, KLHL31, KLHL38, KLKBL4, KLRC4, |
| | KLRK1, KRT6C, KRT72, KRT74, KRT79, KRTAP10-10, KRTAP13-1, |
| | KRTAP13-4, KRTAP19-2, KRTAP20-3, KRTAP2-1, KRTAP21-1, |
| | KRTAP3-2, KRTAP4-1, KRTAP6-2, KRTAP9-1, KYNU, LAMB2L, |
| | LAPTM4B, LARGE, LARP7, LAX1, LCE3E, LCE6A, LCK, LCOR, |
| | LCORL, LDB3, LDHC, LDLRAD3, LDOC1L, LEMD1, LEPREL1, |
| | LGALS8, LHFPL1, LIG1, LILRA4, LINC00158, LINC00364, LINC00456, |
| | LINC00515, LINC00540, LINC00571, LINC00587, LINC00635, |
| | LINC00700, LINC00845, LINC00865, LINC01032, LINC01102, |
| | LINC01122, LINC01265, LINC01269, LINC01280, LINC01298, |
| | LINC01428, LINC01436, LINC01446, LINC01498, LINC01532, |
| | LINC01565, LINC01572, LINGO1, LMO7, LOC100129138, |
| | LOC100129345, LOC100130872, LOC100240726, LOC100329109, |
| | LOC100505795, LOC100506444, LOC100507073, LOC100507537, |
| | LOC100507661, LOC100996671, LOC101926963, LOC101927023, |
| | LOC101927058, LOC101927159, LOC101927244, LOC101927286, |
| | LOC101927358, LOC101927769, LOC101927844, LOC101927901, |
| | LOC101928203, LOC101928441, LOC101928565, LOC101928622, |
| | LOC101928790, LOC101929153, LOC101929512, LOC101929529, |
| | LOC101929563, LOC101929660, LOC102467214, LOC102467223, |
| | LOC102723362, LOC102724053, LOC102724421, LOC102724776, |
| | LOC145814, LOC152024, LOC283867, LOC284688, LOC284950, |
| | LOC285629, LOC285735, LOC338694, LOC390594, LOC404266, |
| | LOC619207, LOC645949, LOC729080, LOC729668, LOC91948, |
| | LOXHD1, LPAR1, LPP, LRIT1, LRRC4C, LRRC7, LRRN1, LRRN2, |
| | LRRTM4, LSAMP-AS1, LVCAT5, LY6G6F, LY86, LYZ, M1AP, MAFK, |
| | MAGEA10-MAGEA5, MAK, MAK16, MAP2, MAP3K4, MAPT, MAS1L, |
| | MAT2B, MARCH1, MBNL1, MBTD1, MCART2, MCART3P, MCMDC2, |
| | MELK, MEPE, METTL14, METTL8, METTL9, MICAL2, MICALL1, |
| | MIDI, MINA, MIR1257, MIR1297, MIR1302-6, MIR1343, MIR155, |
| | MIR2117, MIR218-1, MIR30B, MIR320D2, MIR4471, MIR499, |
| | MIR516A1, MIR518E, MIR518F, MIR520G, MIR524, MIR526B, MIR532, |
| | MIR544A, MIR548A1, MIR54814, MIR549, MIR558, MIR591, MIR602, |
| | MIR603, MIR613, MIR6132, MIR629, MIR646, MIR651, MIR6716, |
| | MIR6840, MIR6880, MIR6890, MIR7641-2, MIR892A, MIR936, MKL1, |
| | MKL2, MLH1, MLIP, MLL2, MLLT4, MMADHC, MMP19, MMP26, |
| | MOBKL3, MOBP, MOV10, MOV10L1, MPDU1, MPP4, MPP6, MPP7, |
| | MRGPRD, MRGPRX2, MRRF, MS4A15, MS4A3, MTCL1, MTMR6, |
| | MUC2, MUSK, MX1, MYH15, MYH2, MYH7, MYLK, MYO16, |
| | MYO18B, MYO7B, MYT1L, NAALADL2-AS2, NACA2, NANP, NAPSB, |
| | NAT16, NCK1, NCK2, NCKAP5, NCOA1, NCOA5, NDST2, NEK11, |
| | NEK7, NEU4, NFASC, NGDN, NGF, NIN, NMD3, NONO, NOTO, NOX4, |
| | NPAS2, NPFFR2, NPHS1, NR1H4, NR2C2AP, NRCAM, NRSN1, |
| | NSMCE2, NUP107, NXPH2, OCA2, ODAM, ODF3L2, OIT3, ONECUT1, |
| | OR10AG1, OR10W1, OR12D2, OR12D3, OR1D2, OR1S1, OR2J2, |
| | OR2T6, OR2V1, OR4C45, OR4N5, OR4S1, OR51D1, OR51G1, OR51L1, |
| | OR5AC2, OR5B12, OR5B3, OR5D18, OR5K2, OR5T2, OR6B1, OR6S1, |
| | OR6X1, OR7G3, OR9G1, OS9, OSBPL3, OSBPL5, OSBPL6, OSBPL8, |
| | OSR1, OTC, OXER1, OXR1, OXTR, PAG1, PAH, PAK3, PALLD, |
| | PAPPA2, PAQR7, PARP1, PATL2, PBK, PBLD, PCDHA2, PCDHB15, |
| | PCDHB2, PCDHB5, PCDHB7, PCDHB9, PCDHGA3, PDC, PDE11A, |
| | PDE4D, PDE9A, PDLIM5, PDS5B, PDZK1, PDZRN3, PEMT, PEX5L, |
| | PGAM2, PGAM5, PGK2, PHACTR3, PID1, PIK3CB, PINK1, PIWIL3, |
| | PIWIL4, PKDREJ, PKHD1L1, PKIA, PKIB, PLA2G2E, PLAG1, PLCB4, |
| | PLCE1-AS1, PLEKHG1, PLP1, PLP2, PLS1, PLXNA4, PMFBP1, |
| | POLDIP2, POLK, POLR3C, PPAP2C, PPM1F, PPP1R3E, PPP2R2C, |
| | PRIM2, PRKG1, PRLH, PRLR, PRPF31, PRPSAP2, PRR16, PRR23B, |
| | PRSS35, PSMD1, PSMD14, PSPC1, PSPN, PTCD2, PTCHD2, PTK2B, |
| | PTN, PTPN4, PTPRK, PTPRZ1, PVALB, PXMP4, R3HCC1L, RAB19, |
| | RAB40A, RAB9B, RAG1, RAI1, RALBP1, RAP1A, RAPGEF5, |
| | RASGEF1A, RBFOX3, RBM44, RBM46, RBM47, RBM6, RBMS3-AS1, |
| | RBMXL2, RBP3, RFPL3, RFPL4B, RGS11, RGS12, RGS3, RHOH, |
| | RIMBP2, RIPPLY2, RLBP1, RLF, RNASE12, RNASEN, RNF133, |
| | RNF19A, RNF2, RNF20, RNF4, RNF6, RNF7, RNMTL1, ROBO2, |
| | RPGRIP1, RPL19P12, RPRD1A, RPS14, RPS15AP10, RPS26, RPTOR, |
| | RUNX1T1, RWDD2B, S100A14, SAG, SAMD4A, SARS, SCAND3, |
| | SCAPER, SCARNA27, SCARNA8, SCMH1, SCOC, SCRN1, SDHAP1, |
| | SEC14L3, SEMA3D, SEMA6A, SENP7, SETD5, SF3A1, SFRS7, SGMS1, |
| | SGMS2, SH2B3, SH2D1A, SH3BP4, SH3D19, SH3KBP1, SHPRH, SIAE, |
| | SIGLEC16, SIL1, SIN3A, SIPA1L2, SLAIN1, SLC10A5, SLC12A5, |
| | SLC12A8, SLC16A1, SLC16A4, SLC17A2, SLC1A4, SLC1A6, SLC22A1, |
| | SLC23A2, SLC24A2, SLC25A30, SLC25A41, SLC26A7, SLC28A3, |
| | SLC30A8, SLC35A3, SLC37A3, SLC39A10, SLC39A4, SLC45A1, |
| | SLC4A11, SLC4A4, SLC5A12, SLC6A15, SLC8A3, SLCO6A1, SLMAP, |
| | SLMO1, SMAD2, SMAD5, SMIM9, SMR3B, SNORA15, SNORA19, |
| | SNORA2B, SNORA59A, SNORD113-2, SNORD113-5, SNORD115-29, |
| | SNORD116-12, SNORD116-2, SNORD47, SNX2, SORBS1, SORBS2, |
| | SORCS1, SOX10, SOX13, SPANXN4, SPATA25, SPATA9, SPECC1L, |
| | SPOCK3, SPRR1A, SPRY1, SPRYD4, SPTBN1, SPTY2D1, SQRDL, |
| | SRMS, SSPN, SSX9, ST18, ST20, ST3GAL3, ST5, ST6GAL2, |
| | STAMBPL1, STARD13, STATH, STK35, STMN4, STOML3, STYK1, |
| | SULT1C3, SUN3, SUPT7L, SUSD5, SV2B, SV2C, SYAP1, SYBU, |
| | SYCP3, SYNE2, SYT14, TAAR1, TAAR2, TAB3, TAF12, TAF1L, TANC1, |
| | TANK, TAS2R13, TAS2R16, TAS2R50, TBC1D5, TCAIM, TCEB3B, |
| | TCHH, TCL1A, TDRD7, TEAD4, TECTB, TENM3, TESPA1, TEX10, |
| | TEX14, TFDP2, TFE3, THEM5, THRB, THSD7B, TIRAP, TLK1, TLR6, |
| | TMC1, TMEM261, TMEM51, TMEM62, TMLHE, TMOD1, TMPRSS11B, |
| | TMPRSS11GP, TNFAIP8L2-SCNM1, TNFRSF19, TNFRSF8, TNFSF11, |
| | TNIP1, TNIP3, TNS3, TNXB, TOX2, TP73, TPD52, TPD52L1, TPRG1, |
| | TPRG1-AS2, TPRXL, TPTE2, TRAF3IP2, TRDN, TRERF1, TRIM15, |
| | TRIM22, TRIM36, TRIM60, TRIM63, TRIP12, TROVE2, TRPC3, TRPC7, |
| | TRPM1, TSGA10, TSHZ1, TSPAN18, TSPAN9, TSPYL1, TSPYL6, |
| | TTC39B, TTC3L, TTC8, TTLL13, TUBA1A, TUBA3D, TUBB, TUBB3, |
| | TUBGCP3, TUG1, TXLNB, TXNDC16, TXNL4A, TXNRD1, UAP1, |
| | UBAP1, UBAP2, UBE2J1, UBE2U, UCHL1, UCP3, UHMK1, UNC84A, |
| | UPF2, USP16, USP25, USP44, UTRN, VANGL1, VCAM1, VN1R1, |
| | VPRBP, VRK1, VRK3, VTCN1, WDR13, WDR17, WDR31, WDR49, |
| | WDR64, WDR76, WNT8B, WSCD2, WSPAR, WTAPP1, XCR1, XIRP1, |
| | YIPF5, YSK4, ZBBX, ZBTB20, ZBTB4, ZCCHC13, ZCCHC5, ZDHHC16, |
| | ZDHHC4, ZFHX3, ZFP2, ZFPM2-AS1, ZHX1, ZHX2, ZMIZ1, ZMYM6, |
| | ZMYND11, ZNF140, ZNF192, ZNF229, ZNF264, ZNF268, ZNF280B, |
| | ZNF280D, ZNF283, ZNF295, ZNF302, ZNF322A, ZNF329, ZNF345, |
| | ZNF350, ZNF366, ZNF395, ZNF415, ZNF438, ZNF469, ZNF516, |
| | ZNF518A, ZNF532, ZNF536, ZNF541, ZNF559, ZNF605, ZNF654, |
| | ZNF664-FAM101A, ZNF691, ZNF704, ZNF713, ZNF730, ZNF775, |
| | ZNF793, ZNF828, ZNF84, ZNF843, ZNF845, ZNF853, ZRANB1, |
| | ZSCAN20, OR2B11 |

4. A Methylation Biomarker Panel

One, two or more genes were selected from 3 subgroups (cluster A, B and C) according to hierarchical clustering of the top 2000 DMPs and created a biomarker panel. The differences of methylation levels in selected genes between pregnancy and non-pregnancy samples were tested by quantitative methylation-specific polymerase chain reaction (qMSP). The present invention further selected SYNE1 from the cluster A; ARID3C, CASR, PDE4C and SLITRK2 from cluster B; and TMEM62 and KCNC2 from the cluster C to validate the pregnancy outcome prediction in IVF. Among the seven selected genes, the AUCs of each single gene ranged from 0.53 to 0.73 in 20 pregnancy and 23 non-pregnancy samples, and ranged from 0.53 to 0.78 in another 32 pregnancy and 37 non-pregnancy samples. To further test the validity of these markers, all the 126 samples were used to estimate the performance of gene combinations by a logistic regression model with 500 times bootstrapping. As demonstrated in Table 5, the AUCs of each single gene ranged from 0.5 to 0.70. Among the selected genes, two genes (SLITRK2 and KCNC2) had only been reported in the nervous system and their role in endometrium was not known. SLITRK2 encodes a transmembrane protein that is involved in the formation and maintenance of synapses. KCNC2 encodes components of voltage-gated potassium channels that are required to maintain the high-frequency firing in neocortical GABAergic interneurons. As for the last two genes, SYNE1 encodes a spectrin repeat-containing protein that anchors the nuclear envelope to the cytoskeleton, which is critical for nuclear positioning. ARID3C encodes a helix-turn-helix transcription factor, implying its role in regulation of gene expression during cell growth, differentiation and development. Multiple markers combined in a biomarker panel may improve diagnostic sensitivity help to optimize the pregnancy outcome prediction in IVF.

TABLE 5
Performance of methylation levels of single gene for
differentiating pregnancy and non-pregnancy samples

| Gene name | AUC |
|---|---|
| ARID3C | 0.67 (0.65-0.72) |
| CASR | 0.64 (0.62-0.79) |
| KCNC2 | 0.70 (0.69-0.75) |
| PDE4C | 0.51 (0.49-0.58) |
| SLITRK2 | 0.70 (0.68-0.74) |
| SYNE1 | 0.57 (0.55-0.62) |
| TMEM62 | 0.50 (0.48-0.57) |

Values are AUC (95% confidence interval). Data are means of AUC (95% confidence interval) calculated by a logistic regression model based on five-fold cross-validation with 500 iterations. AUC: area under the receiver operating characteristic curve.

5. Cross-Validation of Gene Combinations for Predicting Pregnancy Outcomes

To further test the performance of gene combinations of these selected genes in predicting pregnancy outcomes, five-fold cross-validation was performed on all 126 samples, including the discovery and validation sets, to simulate a larger data set that could be used to estimate the out-of-sample performance. In each round of cross-validation, samples were randomly partitioned into five equal-sized subgroups. Four subgroups were used to perform the analysis (the training set) and the remaining subgroup to validate the analysis (the testing set). Compute the AUC scores by performing 5-fold cross-validation. The process was repeated for 5 times with each of the subgroups used exactly once as the validation data. After 500 rounds of five-fold cross-validation, the validation results were logistically regressed, as demonstrated in Table 6. A four-gene panel (including SYNE1, KCNC, SLITRK2, and PDE4C) was established for prediction model. The ROC curve revealed good predicted performance (AUC=0.81). Five-gene combinations or six-gene combinations showed slightly higher AUC (0.81˜0.83).

TABLE 6
Performance of gene combinations for predicting
pregnancy outcomes using cross-validation resampling

| Gene | | | | | |
|---|---|---|---|---|---|
| name | 2 genes | 3 genes | 4 gene | 5 genes | 6 genes |
| | KCNC2 | KCNC2 | KCNC2 | KCNC2 | KCNC2 |
| | | SLITRK2 | SLITRK2 | SLITRK2 | SLITRK2 |
| | | | PDE4C | PDE4C | PDE4C |
| | | | | SYNE1 | SYNE1 |
| | | | | | CASR |
| ARID3C | 0.71 | 0.77 | 0.80 | 0.82 | 0.83 |
| | (0.68-0.75) | (0.73-0.80) | (0.77-0.83) | (0.79-0.85) | (0.80-0.85) |
| CASR | 0.75 | 0.76 | 0.80 | 0.82 | — |
| | (0.71-0.79) | (0.73-0.80) | (0.77-0.83) | (0.79-0.84) | |
| KCNC2 | — | — | — | — | — |
| PDE4C | 0.72 | 0.80 | — | — | — |
| | (0.68-0.75) | (0.77-0.83) | | | |
| SLITRK2 | 0.76 | — | — | — | — |
| | (0.73-0.80) | | | | |
| SYNE1 | 0.71 | 0.76 | 0.81 | — | — |
| | (0.67-0.75) | (0.73-0.80) | (0.78-0.84) | | |
| TMEM62 | 0.71 | 0.79 | 0.80 | 0.81 | 0.82 |
| | (0.67-0.75) | (0.76-0.82) | (0.77-0.83) | (0.78-0.84) | (0.79-0.85) |

Data are means of AUC (95% confidence interval) calculated by a logistic regression model based on five-fold cross-validation with 500 iterations. AUC: area under the receiver operating characteristic curve.

Feature selection is necessary along with model estimation to reduce data dimension and model complexity. The above findings suggested that the methylation levels of selected genes having potential diagnostic usage as biomarkers. Importantly, features combine named as multi-biomarker panel could be an effective approach to improving diagnostic accuracy.

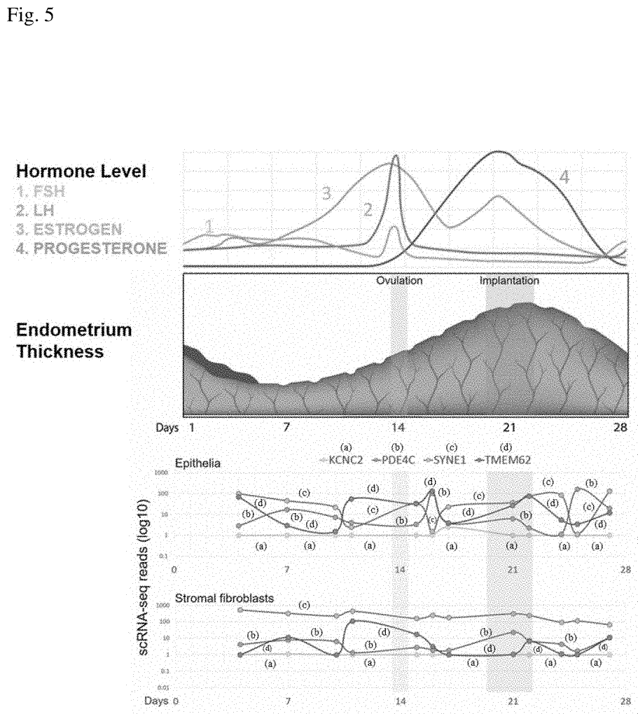

The expression of these selected genes in normal endometrium throughout the menstrual cycle was retrieved from publicly available single-cell RNA-seq data. Only KCNC2, PDE4C, SYNE1, and TMEM62 were available in the database. As illustrated in FIG. 5, the expression of these four genes can be found in both endometrial epithelial cells and stromal fibroblasts. In epithelial cells, the expression levels of PDE4C, SYNE1, and TMEM62 fluctuated immediately after ovulation, but returned swiftly to normal levels and remained relatively stable until the second half of the implantation window. KCNC2 expression showed more stable across the menstrual cycle. In stromal fibroblasts, there was no fluctuation following ovulation, unlike the case in their epithelial counterparts. Only PDE4C and TMEM62 showed transcriptomic changes from the second half of the implantation window, implying the participation of stromal cells in decidualization. The expression levels of KCNC2 in stromal fibroblasts showed stable throughout the menstrual cycle. RNA-seq was widely used to study gene expression changes associated with biological conditions. The RNA-seq data might explain how environmental exposures could modify gene expression. Compared to single-gene biomarkers, the present invention found that cluster-based biomarkers are more robust and effective.

The endometrium undergoes cyclic changes involving cell proliferation, differentiation and degradation, which were driven by steroid hormones (FIG. 5). The conditions of endometrium may be accurately controlled by exogeneous hormones like the preparation of endometrium in artificial cycles for the transfer of frozen embryos. However, it was unlikely to duplicate endometrium between cycles with ovulating ovaries because even the same woman may present different menstrual patterns in natural cycles or respond differently to the same ovarian stimulation protocol in stimulated cycles. Moreover, the regenerated endometrium in each menstrual cycle was constructed by a new colony of progenitor cells, implying a monthly variation of endometrium. The analysis in the present invention using cervical secretions ensured the implantation environment from perturbation, which provided a diagnostic tool to investigate the monthly variation of endometrial receptivity.

Predicting receptivity of endometrium ahead of embryo transfer through quick diagnosis tests would be able to maximize chances of successful pregnancy by saving good embryos to cycles with favorable endometrium. The methylation profile not only provided an objective diagnosis for endometrial receptivity, but also unraveled the molecular involvements in the establishment of pregnancy, which may pave a way for new therapies in endometrial and obstetrical diseases.

Those skilled in the art recognize the foregoing outline as a description of the method for communicating hosted application information. The skilled artisan will recognize that these are illustrative only and that many equivalents are possible.

What is claimed is:

1. A method for identifying a potential biomarker for determining the probability of the success of embryo implantation, comprising:
(1) providing a cervical sample from a female subject;
(2) assaying nucleic acids of the cervical sample to generate a methylation profile comprising 1733 genes listed in Table 4;
(3) calculating a statistical value of at least one gene from the 1733 genes in the methylation profile; and
(4) identifying the at least one gene as a biomarker in the cervical sample for determining the probability of the success of embryo implantation when the statistical value of the at least one gene is higher than a threshold value.

2. The method of the claim 1, wherein the cervical sample is a biological sample taken from the lumen of the cervix, wherein the biological sample comprises secretions, epithelial cells, stromal cells, squamous cells, glandular cells, immune cells, vaginal fluids, vaginal microbiota, mucus molecules or water.

3. The method of claim 1, wherein the cervical sample is obtained from 1 to 5 days before or on the day of the female subject receiving embryo transfer.

4. The method of claim 1, wherein the methylation profile is generated by bisulfite sequencing PCR (BSP), reduced representation bisulfite sequencing (RRBS), whole genome bisulfite sequencing (WGBS), methylated DNA immunoprecipitation sequencing (MeDIP), enzymatic methyl sequencing (EM-Seq), mass spectrometry method, methylation specific PCR, qPCR, PCR, sanger sequencing, next-generation sequencer, methylation chip, methylation chip array, ion torrent sequencer, real-time nanopore sequencing, smaller genomes sequencing, targeted regions sequencing, targeted amplicons sequencing, fiber optical particle plasmon resonance (FOPPR), or changes in transverse proton relaxation.

5. The method of claim 1, wherein the 1733 genes are divided into cluster A comprising 319 genes, cluster B comprising 174 genes and cluster C comprising 1240 genes, wherein the genes in the clusters A, B and C are listed in Table 4.

6. The method of claim 5, wherein the at least one gene is selected from the group consisting of the cluster A, the cluster B and the cluster C.

7. The method of claim 1, wherein the statistical value of the at least one gene is a value of an area under the curve (AUC) calculated by a receiver operating characteristic (ROC) curve.

8. The method of claim 1, wherein the threshold value is 0.7.
